(19) United States
(12) Patent Application Publication
(10) Pub. No.: US 2013/0095160 A1
(43) Pub. Date: Apr. 18, 2013

(54) RESORBABLE POLYSTATIN BIOMATERIALS

(71) Applicants: Gautam S. Ghatnekar, Charleston, SC (US); Wayne H. Kaesemeyer, Augusta, GA (US)

(72) Inventors: Gautam S. Ghatnekar, Charleston, SC (US); Wayne H. Kaesemeyer, Augusta, GA (US)

(21) Appl. No.: 13/612,054

(22) Filed: Sep. 12, 2012

Related U.S. Application Data
(60) Provisional application No. 61/533,722, filed on Sep. 12, 2011.

(51) Int. Cl. A61K 31/765

(52) U.S. Cl. CPC A61K 31/765
USPC 424/402; 528/354; 424/78.38

(57) ABSTRACT

Resorbable biomaterials including polystatin polymers and devices including the resorbable biomaterials are described. Methods of making the resorbable biomaterials and devices including resorbable biomaterials are also described.

### CROSS-REFERENCE TO RELATED APPLICATIONS

This application claims the benefit of U.S. Provisional Application No. 61/533,722 filed Sep. 12, 2011, which is incorporated by reference in its entirety herein.

### BACKGROUND

The use of stents has revolutionized the treatment of coronary heart disease. Hospital operating rooms that previously were devoted to bypass surgery have been converted to interventional suites for percutaneous coronary intervention (PCI) and stent placement.

Since their introduction, stents have evolved through three generations: bare metal, drug eluting and bioresorbable stents. Metal stent construction typically begins with fabrication of a scaffold which is a mesh-like tube of thin wire which props open the lumen of an artery or vein following the performance of a procedure, such as an angioplasty, done to open a vessel narrowed by a disease such as atherosclerosis, without or with accompanying thrombosis. Such scaffolds generally comprise metallic compositions including 316 L stainless steel and cobalt chromium alloy.

Second generation stents which elute drugs placed on the surface of a scaffold for preventing restenosis have also been developed. Drugs can be directly applied to the scaffold of these stents or dissolved in and eluted from biodegradable polymers coated onto metallic scaffolds. Drug eluting stents (DES) have helped prevent and mitigate restenosis. However, the drugs used such as paclitaxel and mTOR or cell cycle inhibitors such as rapamicin and sirolimus can cause endothelial dysfunction, impaired re-endothelization and stent thrombosis. As such, drug-eluting stents have a prolonged requirement for dual antiplatelet therapy (DAPT) with an inherent risk of bleeding, which has been independently associated with early and late mortality. Current guidelines recommend 12 or more months of DAPT following placement of a drug eluting stent. Furthermore, the lack of generally access to the exotic materials required for DAPT has left bare metal stents as the only practical option for treatment in many cases.

Thus, there is a need for stenting and other implantable devices and therapies that deliver statins at a local site, require less antiplatelet therapy or otherwise overcome the limitations of the current compositions and treatments for coronary artery disease.

### SUMMARY

Embodiments of the invention are directed to a resorbable biomaterial including polymeric statins, methods of producing such bioresorbable materials, and methods for using these resorbable biomaterials, including to promote wound healing, reduce scaring, reduce bleeding, and mitigate post-surgical adhesions. In certain embodiments, bioresorbable therapeutically active polymers comprised of statin monomers covalently incorporated into the backbone of polymer scaffolds are disclosed. The polystatin polymers of embodiments may be synthesized from L (-) lactide, glycolide, and a lactone containing statin with an intact lactone ring. In yet other embodiments, everolimus and zotarolimus and other thrombosis reducing drugs are incorporated into the disclosed devices thereby reducing bleeding following device placement and reducing duration and intensity of dual antiplatelet therapy (DAPT) currently required for PCI. The dissolution of the biomaterial when implanted in the injured tissues or wounds may enable the controlled release of the biomaterial to exhibit its pharmacological properties as a drug or prodrug, preferably at least one statin, most preferably a statin possessing plieotropic effects. In addition, the biomaterial may be coated with other pharmacologically active agents, including but not limited to, nitric oxide agonists, antimicrobials, growth factors, anti-inflammatory agents. In some embodiments, the polystatin polymer is extruded to form flexible thin filaments that can be configured to form implants, including but not limited to stents, sutures, membranes, meshes, grafts, synthetic tendons, synthetic ligaments, or the like. In certain embodiments, these polymers are extruded into a scaffold for use as a vascular stent. Additional objects, features, embodiments, and advantages of the invention will become more apparent upon review of the detailed description set forth below.

### DESCRIPTION OF DRAWINGS

FIG. 1 provides a view of a polystatin stent extruded onto a glass rod.

FIG. 2 provides an alternative view of a polystatin stent extruded onto a glass rod.

FIG. 3 provides NMR spectra of a control polymer.

FIG. 4. provides NMR spectra of a polystatin polymer.

### DETAILED DESCRIPTION

Disclosed herein are resorbable biomaterials including polymeric statins, methods of producing such bioresorbable materials, and methods for using these resorbable biomaterials, including to promote wound healing, reduce bleeding reduce scaring, and mitigate post-surgical adhesions et alia. Before the present compositions and methods are described in detail, it is to be understood that they are not limited to the particular compositions, methodologies or protocols described, as these may vary. It is also to be understood that the terminology used in the description is for the purpose of describing the particular versions or embodiments only, and is not intended to limit their scope which will be limited only by the appended claims.

It must also be noted that as used herein and in the appended claims, the singular forms “a”, “an”, and “the” include plural reference unless the context clearly dictates otherwise. Unless defined otherwise, all technical and scientific terms used herein have the same meanings as commonly understood by one of ordinary skill in the art. Although any methods and materials similar or equivalent to those described herein can be used in the practice or testing of embodiments disclosed, the preferred methods, devices, and materials are now described.

Throughout the description, where devices and systems are described as having, including, or comprising specific components, compounds, compositions, or where processes and methods are described as having, including, or comprising specific steps, it is contemplated that, additionally, there are devices, systems, compounds, or compositions of the present invention that consist essentially of, or consist of, the recited components, and that there are processes and methods according to the present invention that consist essentially of, or consist of, the recited processing steps.

Use of the term “about” with respect to any quantity is contemplated to include that quantity. For example, “about 10 mm” is contemplated herein to include “10 mm”, as well as values understood in the art to be approximately 10 mm with respect to the entity described.

It should be understood that the order of steps or order for performing certain actions is immaterial so long as the invention remains operable. Moreover, two or more steps or actions may be conducted simultaneously.

The term “unmodified biocompatible polymer” shall refer to any biocompatible polymer that does not include a statin covalently attached to the polymer. Such unmodified biocompatible polymers include commercially available biocompatible polymers such as poly(glycolide) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy)phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), and the like combinations thereof, as well as any biocompatible polymer not commercially available but that is devoid of covalently attached statin. Unmodified biocompatible polymers encompasses without limitation biocompatible polymers that include other pharmaceutically active agents either incorporated into the structure of the polymer or coating the polymer.

As used herein “self-retaining suture” refers to a suture that may not require a knot in order to maintain its position into which it is deployed during a surgical procedure. Such self-retaining sutures generally include a retaining element or tissue retainer.

As used herein “controlled release” refers to the regulated spatial and temporal release of a pharmacological compound.

As used herein, “subject” includes any organism possessing a wound or tissue injury. A subject can include, but is not limited to, animals, particularly vertebrates, and more particularly mammals (e.g., humans, horses, pigs, rabbits, dogs, sheep).

As used herein, “resorbable” refers to materials that can be broken down in vivo without adversely building up.

As used herein “tissue retainer” refers to a suture element having retainer body projecting from the suture body and a retainer end adapted to penetrate tissue. Each retainer is adapted to resist movement from the suture in a direction other than the direction in which the suture is deployed into the tissue by the surgeon.

As used herein “pharmacologically active” refers to pharmacologic bioactivity inherent in a molecular structure which is exclusive of the pharmacologic activity of agents located in the vicinity but of acting external to the same molecular structure.

There are few treatments for scars, and the available treatments are generally limited to mechanical treatments, for example, occlusive dressings, compression therapy, administration of silicone gel to promote wound hydration, and administration of steroids to the wound site, especially keloids. Scars respond to treatment differently, and some treatment methods administration are contraindicated for certain classes of patients. For example, topical steroid patients are limited to those with bacterial infections, yeast infections, or viral infections affecting the wound site.

Physical barriers such as viscous solutions and hydrogels have been widely used to prevent adhesion formation by limiting tissue apposition during the critical stages of mesothelial repair, and anti-inflammatory agents and fibrinolytic agents have been used in the prevention of adhesions. However, at least 50% of patients treated using these methods still develop significant adhesions. Thus, there is a need for improved methods for treating internal postsurgical adhesions, as well as wound scars and abnormal scars (hypertrophic scars).

Statins are a group of 3-hydroxy-3-methylglutaryl Coenzyme A reductase inhibitors in cholesterol biosynthesis pathway that have been widely used as a cholesterol lowering drug. However, statins have also has been shown to reduce hypertrophic scar formation by inhibiting Connective Tissue Growth Factor (CTGF) when administered at low doses. Similarly, lovastatin and atorvastatin have been administered with the peritoneum to up regulate local fibrinolysis decreasing post operative adhesions. Statins acutely and directly activate endothelial nitric oxide synthase (eNOS) independently of inhibiting HMG Co A Reductase. More recently this pathway has been shown to involve the SR-BI receptor on the endothelium.

Embodiments of the invention include resorbable biomaterials, methods for using the restorable biomaterials, and methods for preparing such resorbable biomaterials, and in certain embodiments, the resorbable biomaterial may be incorporated into the fabrication of implants including, but limited to stents, sutures, membranes, meshes, grafts, and the like. The resorbable biomaterials in such embodiments generally include polystatin polymers and are pharmacologically active for stimulating tissue repair, wound closure, and generally promote wound healing of tissues, organs, surgical wounds, surgical incisions, and the like when administered at or adjacent to the wound. In some embodiments, wounds treated with the resorbable biomaterials of the invention exhibit reduced scar formation and tissue adhesion. In certain other embodiments, the biomaterials prevent excessive bleeding and thrombosis.

Various embodiments of the invention include resorbable biomaterial devices including statin containing polymers that generally have a statin covalently attached to a polymer either by incorporating one or more statin into the main chain of the polymer or by covalently attaching one or more statins to a polymer through functional groups associated with the monomeric units of the polymer. Embodiments are not limited to any specific statin, and all known statins are encompassed by the invention. For example, in some embodiments, one or more statin included in the statin containing polymer may be simvastatin, atorvastatin, cerivastatin, fluvastatin, lovastatin, mevastatin, pitavastatin, pravastatin, rosuvastatin, simvastatin, velostatin, dalvastatin, carvastatin, cefvastatin, or the like. Local delivery of statins to the site of vascular injury can promote re-endothelialization, reduce the risk of stent thrombosis, restenosis and the need for DAPT and its related risk of bleeding post PCI.

In certain embodiments, the statin may be incorporated into a biocompatible polymer by incorporating monomeric units of a statin into a biocompatible polymer. Biocompatible polymers are well known in the art and such biocompatible polymers can be manufactured by well established methods. For example, poly(glycolide) (PGA), poly(L-lactic acid) (PLA), poly(lactide-co-glycolide) (PLGA), poly(L-lactide) (PLLA), poly(D,L-lactide) (P(DLLA)), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)) are biocompatible polymers that are polymerized at least in part by ring opening polymerization. Main chain ester groups created by the ring opening polymerization are susceptible to hydrolysis, which breaks these polymers into non-toxic fragments that can be excreted through the kidneys.

Polylactide is a terpolymer of (a) L(-)lactide, (b) glycolide, and (c) epsilon-caprolactone. Typically, the L(-) lactide component varies from 45-85% by weight. Glycolide varies from 5-50% by weight. And the ε-caprolactone varies from about 15 to 25% by weight. The process of synthesizing polycaprolactone requires the presence of a lactone ring in epsilon-caprolactone. HMG CoA reductase inhibitors, or statins, likewise have structures that can exist in a form with a closed lactone ring.

Certain statins such as, for example, simvastatin and lovastatin include a lactone containing cyclic ring, and other statins include carboxylic acids and alcohol groups that can be joined to form a lactone containing cyclic ring by condensation. By virtue of the lactone, such statins may be incorporated into the main chain of the biocompatible polymers during the ring opening polymerization process. Thus, as hydrolysis breaks down the biocompatible polymer the statin incorporated into the main chain is released and is free to stimulate healing. While embodiments are not limited to particular polymers including main chain statins, in an illustrative embodiment, simvastatin may be incorporated into a PLGA biocompatible polymer during ring opening polymerization as illustrated in Formula I:

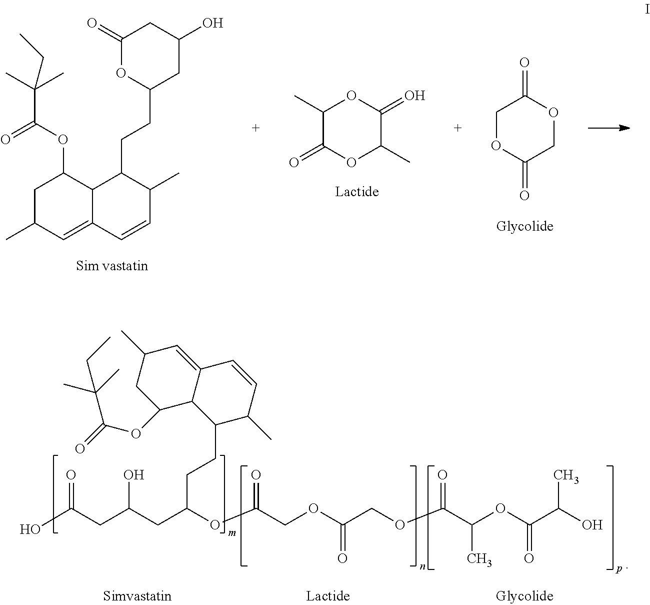

In such embodiments, m, n, and p can be any integer and represent the total number of units for each simvastatin, lactide, and glycolide monomer. The skilled artisan will understand that the monomeric units derived from each monomer are randomly distributed throughout the polymer.

The amount of each monomeric unit in such embodiments can vary and may depend on the desired properties for the statin containing polymer. In some embodiments, statin containing polymers may include one or more statin that is polymerized with a single monomer, such as, lactide, glycolide, or ε-caprolactone to create a statin/lactide, glycolide, or ε-caprolactone copolymer. In such embodiments, the statin may be about 0.5 wt % to about 50 wt. % of the total copolymer, and the lactide, glycolide, or ε-caprolactone may make up the remainder of the copolymer (i.e., about 50 wt. % to about 99.5 wt. % of the total polymer. In embodiments, such as those exemplified in Formula I in which a terpolymer is created, the statin may be about 0.5 wt % to about 50 wt. % of the total copolymer, and the concentration of the remaining monomers may be determined based on the concentration of monomers in known copolymers. For example, in some embodiments, a statin containing polymer prepared from a terpolymer of, for example, lactide, glycolide, and a lactone containing statin may include lactide at a concentration of about 45 wt. % to about 85 wt. % of the total polymer, glycolide about 5 wt. % to about 50 wt. % of the total polymer, and lactone containing statin may, generally, be about 5 wt. % to about 30 wt. % of the total polymer. In other exemplary embodiments, the lactide may be about 55 wt. % to about 75 wt. % or about 60 wt. % to about 70 wt. % of the total polymer, the glycolide may be about 10 wt. % to about 30 wt. % of the total polymer, and the lactone containing statin may be about 15 wt. % to about 25 wt. % of the total polymer. In all such embodiments, the skilled artisan may introduce more or less statin to provide resorbable biomaterials having different dosage.

In various embodiments, the ratio of statin, lactide and glycolide in the terpolymer may be about 70-15-15, about 70-10-20, about 70-20-10, about 80-10-10, about 80-15-5, about 80-5-15, about 60-20-20, about 60-10-30, about 60-30-10, about 60-15-25, about 60-25-15, about 90-5-5, or any value in ranges thereof.

In other embodiments, the statin may be covalently attached to a biocompatible polymer through functional groups associated with the biocompatible polymers. For example, in some embodiments, a statin may be coextruded with a commercially available biocompatible polymer to produce a biocompatible polymer having statin pendant or end groups. For example, many commercially available biocompatible polymers can include carboxylic acid or ester end groups including, but are not limited to, poly(glycolide) (PGA), poly(L-lactic acid) (PLA), poly(lactide-co-glycolide) (PLGA), poly(L-lactide) (PLLA), poly(D,L-lactide) (P(DLLA)), poly(ethylene glycol) (PEG), poly(ε-caprolactone) (PCL), montmorillonite (MMT), poly(L-lactide-co-ε-caprolactone) (P(LLA-CL)), poly(ε-caprolactone-co-ethyl ethylene phosphate) (P(CL-EEP)), poly[bis(p-methylphenoxy)phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), poly(ethylene-co-vinyl alcohol), and combinations thereof. Statins that do not include a lactone ring or statins that have been saponificated to break the ester of lactone ring creating a carboxylic acid and an alcohol group may be covalently attached to such biocompatible polymers through these carboxylic acid of ester end groups of the biocompatible polymer or through hydrolysis of an ester in the main chain. For example, in some embodiments, a non-lactone containing statin such as, pravastatin, may be covalently attached to a biocompatible polymer such as, PLGA, as illustrated in Formula II:

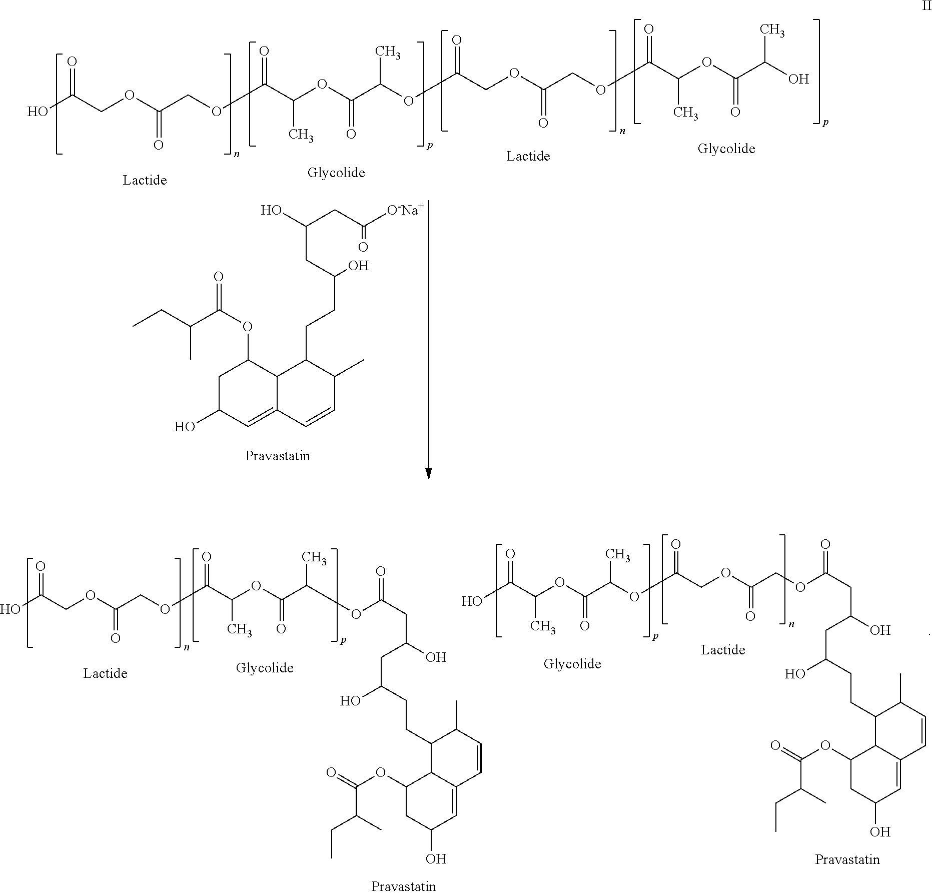

As above, in Formula II, n and p represent an integer providing the number of individual monmeric units of each component on either side of the hydrolyzed ester. The degree of hydrolysis and, thus, the amount of hydrolysis that occurs along the biocompatible polymer in such embodiments may be controlled based on the amount of statin added and the time period over which the reaction is allowed to continue. Without wishing to be bound by theory, in various embodiments, the amount of statin provided may be about 0.5 wt. % to about 25 wt. % of the total polymer to maintain polymer molecular weight that is appropriate for further processing.

In some embodiments, statin containing polymers produced to either incorporate the statin into the main chain of a biocompatible polymer as exemplified by Formula I or by providing statin end groups as exemplified by Formula II, can be combined with a second biocompatible polymer that does not include a statin to create a polymer blend. For example, in various embodiments, a statin containing polymer prepared as described herein may be combined with commercially available biocompatible polymers such as those described herein that have not been modified in any way. Without wishing to be bound by theory, including unmodified biocompatible polymers with the statin containing biocompatible polymer may produce a polymer blend with physical properties that are at least comparable to the unmodified biocompatible while still providing local deliver of the statin to the wound. Such blends may be prepared at any ratio and may include two or more unmodified biocompatible polymers and/or two or more statin containing biocompatible polymers. For example, in some embodiments the ratio of unmodified biocompatible polymers to statin containing polymers may be 1:1, 2:1, 3:1, 5:1, 10:1, 20:1, 50:1, 1:2, 1:3, 1:5, 1:10, 1:20, 1:50 or any ratio between any two of these values.

In certain embodiments, one or more additional pharmaceutically active agents can be added to a composition including a statin containing biocompatible polymer, either by including the pharmaceutically active agent in the formulation of the statin containing biocompatible polymer such that the pharmaceutically active agent becomes enmeshed in the matrix of the polymer, or by coating structures or devices made from the statin containing biocompatible polymers with a pharmaceutically active agent. Embodiments are not limited to particular pharmaceutically active agents. For example, in some embodiments, a nitric oxide agonist may be included with the statin containing biocompatible polymer either by incorporating the nitric oxide agonist into the statin containing polymer or coating a structure or device with the nitric oxide agonist to potentiate the effect of the statin during resorption.

In other embodiments, the pharmaceutically active agents based on polynucleotides, polypeptides, oligonucleotides, gene therapy agents, nucleotide analogs, nucleoside analogs, polynucleic acid decoys, therapeutic antibodies, anti-inflammatory agents, blood modifiers, thrombolytics, anti-platelet agents, anti-coagulation agents, immune suppressive agents, anti-neoplastic agents, anti-cancer agents, anti-cell proliferation agents, anti-microbial, antibiotics, anti-fungal agents, and the like or combinations thereof may be included with the statin containing biocompatible polymer.

More specific examples of pharmaceutically active agents that can be included in the statin containing biocompatible polymers include, but are not limited to, thrombin inhibitors; antithrombogenic agents; thrombolytic agents; fibrinolytic agents; vasospasm inhibitors; calcium channel blockers; vasodilators; antihypertensive agents; antimicrobial agents, such as antibiotics (such as tetracycline, chlortetracycline, bacitracin, neomycin, polymyxin, gramicidin, cephalexin, oxytetracycline, chloramphenicol, rifampicin, ciprofloxacin, tobramycin, gentamycin, geldanamycin, erythromycin, penicillin, sulfonamides, sulfadiazine, sulfacetamide, sulfamethizole, sulfisoxazole, nitrofurazone, sodium propionate), antifungals (such as amphotericin B and miconazole), and antivirals (such as idoxuridine trifluorothymidine, acyclovir, gancyclovir, interferon); inhibitors of surface glycoprotein receptors; antiplatelet agents; paclitaxel, mTORinhibitors; as well as cell cycle inhibitors (e.g., rapamicin and sirolimus), sirolimus derivatives (e.g., everolimus and zotarolimus); antimitotics; microtubule inhibitors; anti-secretory agents; active inhibitors; remodeling inhibitors; antisense nucleotides; anti-metabolites; antiproliferatives (including antiangiogenesis agents); anticancer chemotherapeutic agents; anti-inflammatories (such as hydrocortisone, hydrocortisone acetate, dexamethasone 21-phosphate, fluocinolone, medrysone, methylprednisolone, prednisolone 21-phosphate, prednisolone acetate, fluorometholone, betamethasone, triamcinolone, triamcinolone acetonide); non-steroidal anti-inflammatories (such as salicylate, indomethacin, ibuprofen, diclofenac, flurbiprofen, piroxicam); antiallergenics (such as sodium chromoglycate, antazoline, methapyriline, chlorpheniramine, cetrizine, pyrilamine, prophenpyridamine); anti-proliferative agents (such as 1,3-cis retinoic acid); decongestants (such as phenylephrine, naphazoline, tetrahydrazoline); miotics and anti-cholinesterase (such as pilocarpine, salicylate, carbachol, acetylcholine chloride, physostigmine, eserine, diisopropyl fluorophosphate, phospholine iodine, demecarium bromide); antineoplastics (such as carmustine, cisplatin, fluorouracil); immunological drugs (such as vaccines and immune stimulants); hormonal agents (such as estrogens, estradiol, progestational, progesterone, insulin, calcitonin, parathyroid hormone, peptide and vasopressin hypothalamus releasing factor); immunosuppressive agents, growth hormone antagonists, growth factors (such as epidermal growth factor, fibroblast growth factor, platelet derived growth factor, transforming growth factor beta, somatotropin, fibronectin); inhibitors of angiogenesis (such as angiostatin, anecortave acetate, thrombospondin, anti-VEGF antibody); dopamine agonists; radiotherapeutic agents; peptides; proteins; enzymes; extracellular matrix components; ACE inhibitors; free radical scavengers; chelators; antioxidants; anti-polymerases; photodynamic therapy agents; gene therapy agents; and other therapeutic agents such as prostaglandins, antiprostaglandins, prostaglandin precursors, and the like. In still other embodiments the additional pharmaceutically active agent may be a combination of these agents and/or a nitric oxide agonist.

In certain other embodiments, the biomaterial includes active agents that avoid endothelial dysfunction, impaired re-endothelization and stent thrombosis that may be associated with rapamycin and sirolimus treatment. Without excluding such active agents, the device may include everolimus and zotarolimus as the active agent. In some embodiments, the active agent may be present in, for example, one part for every ten parts of polymer. Examples of ratios in which the active agent may be present in a composition having the terpolymer and the active agent include, but are not limited to, 5:1, 7.5:1, 10:1, 15:1, 20: 1, 25:1, or any value in range between any two of these values.

Embodiments include methods for preparing statin containing biocompatible polymers described above. For example, in some embodiments, terpolymers lactide, glycolide, and a lactone containing statin can be synthesized in accordance with the method described by Goldberg, U.S. Pat. No. 5,085,629, which is hereby incorporated by reference in its entirety. Methods encompassed by the invention include statins containing lactone rings such as simvastatin and lovastatin as well as other statins which can be modified to include a lactone ring or subjected to a condensation reaction to create a lactone ring from free carboxylic acids and alcohol groups on the statin. Such embodiments include the use of any ring containing monomers, for example, D or L lactide or combinations thereof, and D or L glycolide or combinations thereof may be used as monomeric starting materials to synthesize statin containing biocompatible polymers in which the statin is incorporated into the main chain of the polymer. As discussed above, these monomers may be used only to produce statin containing copolymers or in combinations to create statin containing terpolymers. In other embodiments, D or L ε-caprolactone or a combination thereof may be used as a starting material for the synthesis of statin containing biocompatible polymers, and the ε-caprolactone can be used alone or in combinations with lactide or glycolide monomers to create various terpolymers. In additional embodiments, other monomers including, but not limited, monomers containing vinyl, acetyl, carboxylic acid, ester, epoxy, urethane, ketone, and the like groups that may be used to make biocompatible polymers may be incorporated into reaction mixtures including statins to create various statin containing biocompatible polymers.

In some embodiments, methods for preparing statin containing biocompatible polymers may include providing a catalyst to a reaction mixture including one or more statins and monomers used to create statin containing biocompatible polymers. Catalysts for ring opening polymerization are well known in the art, and any catalyst known and useful for such reactions can be used in conjunction with the methods described herein. For example, in various embodiments, the metal carboxylate catalyst or other catalysts such as metal oxides, and metal alkoxides may be used. Known ring opening polymerization catalysts include carboxylates, oxides, and alkoxides of tin, zinc, and aluminum, all of which may be used in embodiments of the invention. In other embodiments, the reaction may include one or more accelerators, initiators, branching agents, and the like, and combinations thereof.

Ring opening polymerization may be carried out in a reactor under inert atmosphere such as nitrogen or argon. In some embodiments, ring opening polymerization can be performed by solution polymerization in an inactive solvent such as benzene, toluene, xylene, cyclohexane, n-hexane, dioxane, chloroform, or dichloroethane, and in other embodiments, the ring opening polymerization may be carried out by bulk polymerization. The reaction temperature can be about ambient temperature to about 250° C., and in polymerization may be carried out for from about 0.5 to about 75 hours. In particular embodiments, the ring-opening polymerization can be carried out at an elevated temperature of about 100° C. to about 150° C., or about 120° C. to about 130° C., and polymerization may be carried out for about 1 hour to about 20 hours or about 15 hours to 20 hours. In some embodiments, the polymer prepared by ring opening polymerization may be endcapped to limit the size of the polymer or to provide a precursor for block copolymer synthesis.

Embodiments further include incorporating the resorbable biomaterial into a device or implant and devices and implants including the resorbable biomaterial. For example, in some embodiments, an implant such as, but not limited to, a suture, a membrane, a mesh, a graft, and the like may be made from resorbable biomaterial including polystatin polymers. In certain embodiments, resorbable biomaterial including polystatin polymers may be incorporated into such implants. Such implants may be useful for promoting healing and mitigating deleterious adhesions in the repair of injured tissues and organs. In such embodiments, the implants or devices may be completely resorbable.

In other embodiments, statin containing biocompatible polymer may be incorporated into an existing biocompatible polymer as exemplified in Formula II by extruding a biocompatible polymer with one or more statins. In such embodiments, extrusion may be carried out by injecting one or more statins into an extruder holding a biocompatible polymer. Extruders generally include screws which mix the components before forcing these mixture through a die. In such embodiments, the biocompatible polymer may be melted by heating the polymer in the extruder before adding the statin.

In certain embodiments, a statin containing biocompatible polymer prepared by either incorporating the statin into the main chain of a biocompatible polymer or attaching the statin end groups to a biocompatible polymer may be combined with an unmodified biocompatible polymer by, for example, melt mixing or extrusion. Such methods are well known in the art and can be employed by any skilled artisan.

The statin containing biocompatible polymers and polymer mixtures containing statin containing biocompatible polymers may be used to coat existing devices, spun into fibers, yarns or filaments, or prepared as membranes, fabrics, meshes, or mesh-like structures. In some embodiments, the statin containing biocompatible polymers may be made into fibers, yarns, or filaments that can be interconnected or otherwise associated with one another by, for example, weaving or knitting the fibers, yarns, or filaments to form fabrics that may or may not include mesh-like openings.

Embodiments provide methods for fabricating devices from the various statin containing biocompatible polymers and mixtures of statin containing biocompatible polymers prepared as described above. For example, in some embodiments, stress loads above the elastic deformation point can be applied to the biomaterial, causing permanent elongation, and extrusion of one or more monofilaments that may vary in size, shape, length, and diameter. In other embodiments, the resorbable biomaterial extrusions may be converted into filaments by a conventional melt spinning process. In a melt spinning process, the biomaterial may be melted, spun from the melt into filaments, and stretched and annealed. Such monofilaments can be then be braided, twisted, spun, woven, knitted, laminated, and such to create fabrics, membranes, meshes, mesh-like structures or other implants. In particular embodiments, the materials created by such processes may be used as sutures, synthetic tendons, synthetic ligaments, coverings for wounds, adhesion barriers, and the like. In still other embodiments, the statin containing biocompatible polymers or mixtures of statin containing biocompatible polymers may be extruded and used as coatings for existing devices. Methods for coating devices are well known in the art, and the skilled artisan can choose from any such method depending on the device coated to apply a coating of the statin containing biocompatible polymer.

In other embodiments, films, membranes, or meshes of statin containing biocompatible polymers or mixtures of statin containing biocompatible polymers can be prepared by solvent transfer methods. Such films or membranes may, generally, have a unitary structure of substantially uniform thickness throughout. In some embodiments, such films or membranes may have gaps or openings that can provide for a mesh or mesh-like structure. In some embodiments, the films, membranes and meshes prepared by such methods may be used alone or layered or laminated with other films, membranes, or meshes prepared by the same method and including statin containing biocompatible polymers or mixtures of statin containing biocompatible polymers, or fabrics or membranes prepared from fibers or filaments of statin containing biocompatible polymers or mixtures of statin containing biocompatible polymers prepared as described herein. In still other embodiments, films, membranes, or meshes may be layered or laminated with fabrics, membranes, or meshes prepared from unmodified biocompatible polymers.

In some embodiments, individual filaments of the statin containing biocompatible polymers may have similar physical and mechanical characteristics as unmodified biocompatible polymers having a similar composition. Thus, devices including fibers, yarns, filaments, membranes, fabrics, meshes, or mesh-like structures prepared from the fibers, yarns or filaments, or devices prepared as membranes, fabrics, meshes, or mesh-like structures may be used alone. In other embodiments, fibers, yarns, filaments, or prepared as membranes, fabrics, meshes, or mesh-like structures prepared from unmodified biocompatible polymers may be incorporated into such materials including statin containing biocompatible polymers to improve physical or mechanical properties of the material, such as prevention of stress fracturing in stents or other mechanical properties that may be needed.

Similarly, in some embodiments, the statin containing biocompatible polymers may have similar resorption behavior and uniformity of resorption following the use in a patient, and the resorption may occur at a sufficient rate to release an effective amount of statin. As such, the statin containing biocompatible polymers may be used alone to effectuate treatment. In other embodiments, the material including the statin containing biocompatible polymers may also include unmodified biocompatible polymers which slow the resorption and release of the statin from the statin containing biocompatible polymers or reduce the amount of statin released during resorption.

In still other embodiments, the material including fibers, yarns, filaments, membranes, fabrics, meshes, or mesh-like structures, including stents, prepared from the statin containing biocompatible polymers may be layered or laminated with additional materials including statin containing biocompatible polymers, additional material including unmodified biocompatible polymers of combinations thereof. In various embodiments, each layer of a multi-layered membrane, mesh, or mesh-like structure may be bonded or otherwise connected to adjoining layers, and in some embodiments, the individual layers of a multi-layered structure may or may not be bonded or otherwise connected to adjoining layers. These layered structures can include one or more layers of statin containing biocompatible polymers and none or one or more layers of unmodified biocompatible material. These layered structures may be designed to provide materials that resorb and/or release statin at particular rates or that have particular mechanical or physical properties. In some embodiments, the layered or laminated structures may provide materials that create partial structures as they resorb that help support a wound as it heals by providing materials that have different resorption behavior.

The fabrics, membranes, meshes, and mesh-like structures of various embodiments may be flexible or compliant, such that they can be contoured to fit the surface of a complex organ. In other embodiments, the fabrics, membranes, meshes, and mesh-like structures can be substantially non-flexible or non-compliant, in whole or in part. In still other embodiments, fabrics, membranes, meshes, or mesh-like structures can be substantially flat or such fabrics, membranes, meshes, or mesh-like structures can be manufactured to exhibit curvature and/or non-planar features such as, for example, convex, concave, or other three-dimensional shapes. To create such structures, fibers of unmodified biocompatible materials may be included in the fabrics, membranes, meshes, and mesh-like materials that increase the rigidity of the device as a whole.

According to need, the molecular weight of statin containing terpolymer can be adjusted. At a molecular weight in the 5,000 to 10,000 range, a softened gel state can be created which melts at slightly above room temperature. At higher molecular weights, such as for example, the range of 50,00 to 100,000, a stenting device can be created that has sufficient radial strength sufficient to preventing strut fracture as can be common among currently available devices. In various embodiments, the molecular weight of the statin containing terpolymer may be in the range of about 5,000 to 15,000, about 15,000 to 25,000, about 25,000 to 50,000, about 50,000 to 100,000, about 100,000 to 200,000, about 200,000 to 500,000, about 500,000 to about 1,000,000, or any range between thereof.

Certain embodiments are directed to self retaining sutures having one or more barbs arranged along the periphery of a filament prepared from the statin containing biocompatible polymers and mixtures of statin containing biocompatible polymers of various embodiments. Barbs can be formed by any known method including, for example, nano-machining, laser ablation, or chemical etching. In some embodiments, self-retaining monofilament sutures can be generated by using a laser as the tissue retainer cutting device. A variety of different wavelength lasers for ablation of materials can be used for nano-machining of statin containing biocompatible polymers. Selection of appropriate pulsing of the laser beam and also a polymer with an appropriate glass transition temperature can be used to adjust the dimensions and characteristics of the tissue retainer formed from the polymer. In some embodiments, immersion of the suture in an organic solvent prior to and/or during laser ablation can be used to control the tissue retainer size and/or depth (about 100 nanometers to about 100 micrometers) that the tissue retainer is etched in the suture.

In particular embodiments, the statin containing biocompatible polymers can be used to make small self-retaining monofilament filaments similar in size to, for example, U.S.P. 7/0, 8/0, 9/0, 10/0 and 11/0 sutures, and in other embodiments, the statin containing biocompatible polymers can be blended with unmodified biocompatible polymers to make small self-retaining monofilaments similar in size to, for example, U.S.P. 7/0, 8/0, 9/0, 10/0 and 11/0 sutures. In still other embodiments, filaments prepared from the statin containing biocompatible polymers can be braided together to produce self-retaining sutures and such sutures can be sized equivalent to U.S.P. monofilament 9/0 and 10/0, but both larger and smaller filament sizes are also envisioned. In other embodiments, statin containing biocompatible polymers can be made into a sewing or suture thread. The suture of such embodiments may be monofil, multifil, braided monofil, or pseudomonofil, surgical suture. The sewing or suture thread form can be further configured to form implants, such as membranes, meshes, synthetic tendons, synthetic ligaments, and the like.

In some embodiments, the statin containing biocompatible polymers can be formed into a flat band or formed into a cord with a substantially round cross-section to serve as synthetic tendons and ligaments. In such embodiments, a braid or cord can be formed from at least two filaments, and in certain embodiments, filaments or fibers of various sizes can be used to construct the multifilament braid. In other embodiments, a braided suture can be made with and without a braid core. In embodiments including a braid core, braid core can be a single monofilament core, a collection of parallel multi-filaments (i.e., a core comprising many small monofilament fibers having little or no twist), twisted multifilament core, and/or a braided multifilament core.

In certain embodiments, the devices including filaments, fibers, strands, sutures, fabrics, membranes, meshes, and mesh-like structures prepared from statin containing biocompatible polymers or mixtures including statin containing biocompatible polymers can be coated with one or more additional therapeutic agents. Embodiments are limited to particular pharmaceutical agents and may include pharmaceutically active agents such as polynucleotides, polypeptides, oligonucleotides, gene therapy agents, nucleotide analogs, nucleoside analogs, polynucleic acid decoys, therapeutic antibodies, anti-inflammatory agents, blood modifiers, anti-platelet agents, anti-coagulation agents, immune suppressive agents, anti-neoplastic agents, anti-cancer agents, anti-cell proliferation agents, anti-microbial, antibiotics, anti-fungal agents, nitric oxide agonists, and the like and combinations thereof exemplified above. Methods for coating such devices are well known in the art, and the methods encompassed by the invention include all such methods.

Embodiments include methods for treating a wound by applying a device prepared from statin containing biocompatible polymers described herein to a wound. The devices and material described herein may be used on any organ to reduce or eliminate scarring. Devices and materials including statin containing biocompatible polymers or mixtures of statin containing biocompatible polymers can be applied to any organ including, but not limited to, the skin, heart, brain, stomach, intestine, spine, spinal cord, teeth, nose, ears, throats, lungs, uterine walls, penis, vagina, blood vessels, eyes, and the like. The step of applying may be carried out in any way. For example, in some embodiments, a fabric, membrane, mesh, or mesh-like structures including statin containing biocompatible polymers can be laid over a wound or adhered to a wound using adhesive tapes or other adhesives useful in the medical arts. In other embodiments, devices and materials encompassed by the invention may be inserted through or between tissues to secure or provide temporary fixation of injured tissues to one another. The injury may be the result of a surgical incision or a wound and may be secured or fixed using sutures or other fabrics or materials prepared from statin containing biocompatible polymers or mixtures of statin containing biocompatible polymers. In other embodiments, fabrics or materials including statin containing biocompatible polymers or mixtures of statin containing biocompatible polymers may be layered over wounds or incisions that have been sutured with sutures prepared from unmodified biocompatible polymers. In still other embodiments, cords or yarns prepared from statin containing biocompatible polymers or mixtures of statin containing biocompatible polymers may be used to attach tendon to bone, re-establish structural integrity of organs such as the skin with minimal scarring, or prevent adherence which may scar. In particular embodiments, the statin containing biocompatible polymers may be incorporated into braided, barbed self-retaining sutures.

Additionally, devices such as vascular stents can be manufactured from the biopolymers disclosed herein. Unlike the case of drug eluting stents where agents are dissolved in biodegrable polymers and eluted from the surface of scaffolds and subject to both luminal and abluminal losses, the disclosed polystatins are tethered to the scaffold and released with scaffold bioresorption. This process of drug exudation will enhance drug receptor interaction relative to the lesser efficient process of drug elution. As such, a scaffold is not only bioresorbable but can also, in and of itself, act as a drug.

Similarly, wherein the embodiments include statins that are incorporated in the backbone of a bioresorbable polymer, the device itself is incorporated into the vessel wall and becomes a system for targeting bioactivated statin monomers to cell surface receptors and intracellular signaling pathways that are involved in the inflammatory processes leading to thrombosis and restenosis. Indeed, in certain embodiments, a second generation mTOR agent can incorporated onto or into the device and be thereby be eluted, creating a dual drug delivery system for treatment. As a result, the duration and intensity of DAPT following interventional procedures can be markedly reduced in order to reduce morbidity and mortality from bleeding.

### EXAMPLES

### Example 1

### Bioresorbable Polystatin Synthesis

A mixture of 11.06 g lactide (79 mmol, 4.3×), 2.13 g glycolide (18.4 mmol, 1×), 1.68 g caprolactone (14.7 mmol, 0.8×), 8.94 g lovastatin (22.1 mmol, 1.2×), 401 mg dodecanol (2.14 mmol, 0.12×) and 1.2 g Sc(OTf)₂ (2.6 mmol, 0.02 mol % of total monomers) in a flask was degassed through a septum with a needle for 10 minutes. The vacuum was replaced with argon and the flask was sealed with a stopper wired in place. The mixture was then shaken in 100° C. oil bath until all of the solids dissolved into a homogenous solution. The flask was allowed to rest in the hot oil overnight. After 18 hours, it formed a dark mass. This was cooled to room temperature and dissolved in dichloromethane (DCM) (˜50 ml), followed by precipitation into 1 1 hexane. After stirring, the supernatant was decanted and the hexane replaced several times, with ˜20 minutes of vigorous stirring each time. The polymeric precipitate was dried in vacuo at room temperature overnight to give ˜16 g dark, viscous material. It was estimated that the lovastatin polymer's molecular weight was between 5,000 and 10,000. The polymer and control were characterized by 1H NMR.

Standard laboratory reagents and solvents were purchased from Alfa Aesar and Fisher Scientific and were used without further purification. 1H NMR spectra were acquired with a Bruker Avance 500 MHz spectrophotometer.

It was found that epsilon caprolactone can be substituted, partially or fully, with either simvastatin or lovastatin in polystatin synthesis with opening of the statin lactone ring resulting in incorporation of monomeric statins in the polymeric backbone via covalent bonding. Lovastatin was used in further experiments due to its vitro potency on activating eNOS. Lovostatin is more efficient than HDL at generating EDNO (endothelium derived nitric oxide) via activation of the SR-BI receptor.

### Example 2

### Stent Fabrication and Extrusion

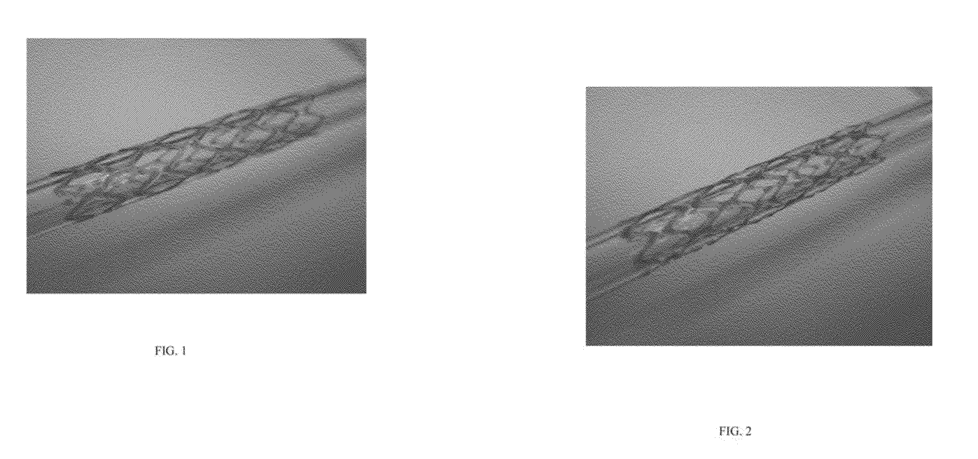

Stents (FIG. 1 and FIG. 2) were fabricated using a Rapid Stent Fabrication (RSF) system developed by 3D Biotek. The RSF system includes an extrusion system and a 4-axis motion system. A computer program controls the 3D extrusion system, and the 4^{th} rotational axis is used to collect the extruded polymer from the extruder. It was possible to make the polymer stents directly from polymer pellets or powder. The material was fed into the extruder chamber through which heat was applied to melt the material in the molten state. At the same time, the extrusion motor was powered to extrude the molten material through a nozzle tip of diameter ranging from 50 to 500 microns. The extruded material was in the form of fiber that was collected on the rotating rod of a diameter ranging from 2 to 8 mm. The combination of the linear motion of the extruder and the rotational motion of the 4th axis (termed “toolpath” herein), guided the extruded fiber deposition on the rod to form various patterns of the stent. The fiber diameter and the pattern dictated the mechanical properties of the stent.

A stent of 40 mm long was fabricated in less than 5 minutes. This RSF system has been successfully used to fabricate the coronary and peripheral stents from different polymers including poly(ε-caprolactone) (PCL), poly-L-lactide (PLLA), poly-D-lactide (PDLA), polyglycolide (PGA), poly(lactide-co-glycolide), polystyrene and polyurethane. The RSF system was used to fabricate stents and tubular scaffolds with complicated pattern designs, and the strut thickness was as thin as 50 micrometers, which renders the scaffolds with extra flexibility and softness.

Compared with laser cutting method, which is the most widely used method for stent fabrication, the RSF system had the additional advantages of high production efficiency and cost saving. Considering laser cutting is a two-step process with formation of tubular structure followed by laser cutting, and up to 90% of the materials are wasted, the RSF technique w a one-step process and 100% of materials are used for the stent.

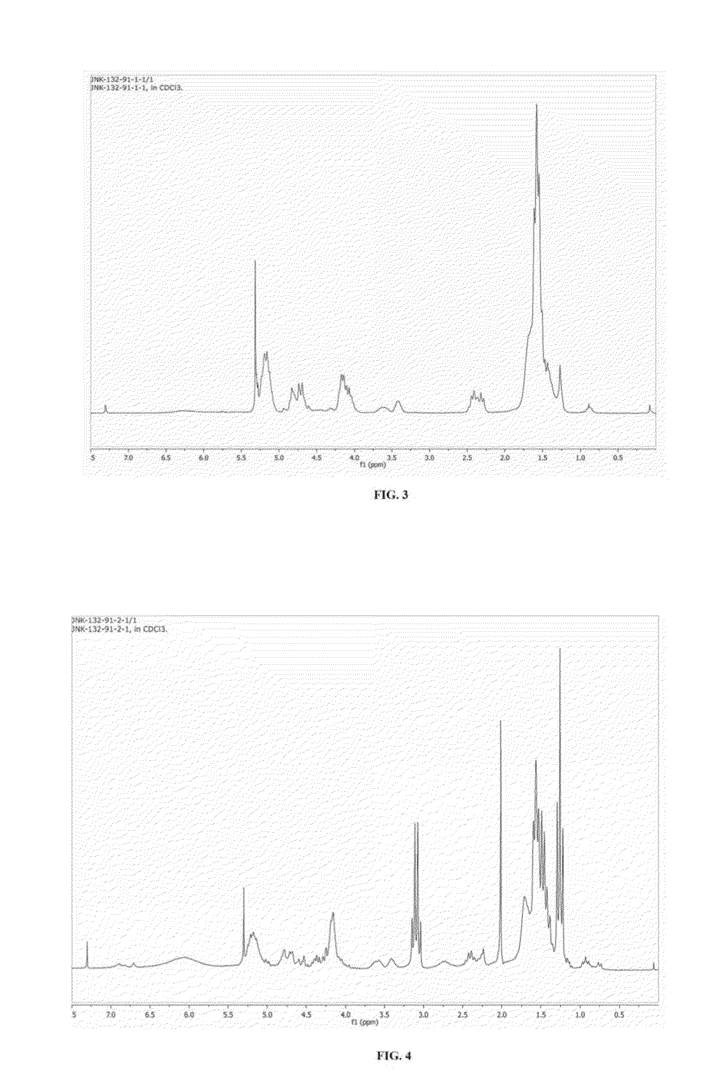

Stent scaffolds were manufactured by extruding bioresorbable polymer in which half the usual content of epsilon caprolactone had been replaced by lovastatin. It was comprised of lactide—glycolide—caprolactone—lovastatin, 60-15-10-15 parts by weight, respectively. The polymer was made by reacting its components at 100° C. in the presence of an alcohol initiator and a scandium catalyst for 18 hours. This was followed by a purification step and characterization by nuclear magnetic resonance spectroscopy (NMR). NMR studies (FIG. 3 and FIG. 4.) of the polymer were consistent with covalent incorporation of lovastatin in the polymer's backbone.

### Example 3

### Dissolution Studies

A scaffold weighing 100 mg in which 60% of ε-caprolactone was replaced with lovastatin. The scaffold delivered 12 mg (or 10(−5) M lovastatin), an amount previously shown to be maximally effective in vitro at eNOS activation. As such, local concentrations of such an amount are not achievable via oral dosing because such systemic concentrations in a patient from orally doses would be highly hepatoxic and myotoxic. However, it should be noted orally dosed statins could be used concurrently with the polystatin scaffolds since the local vessel injury is the only site requiring higher statin dosing which, when released from the scaffold during bioresorption, would not become available to the circulation.

### Example 4

### Terpolymer Compositions

Various terpolymer compositions will be obtained using the method of Example 1 as illustrated in table below.

| Example | L(−) lactide | glycolide | ε-caprolactone | lovastatin |
|---|---|---|---|---|
| a | + | + | + | + |
| b | + | − | − | + |
| c | − | + | − | + |
| d | − | − | + | + |
| e | + | − | + | + |
| f | + | + | − | + |
| g | − | + | + | + |

### Example 5

### Stent Testing

For clinical testing, the polymers will be modified so that its chain length is extended and the molecular weight is in the 100,000 range to increase the radial strength ratio and, thus, minimize the risk of strut fracture. The dissolution products will be studied by HPLC-MS to identify the composition which releases the highest percentage of statin in the open ring configuration that is capable of activating nitric oxide production by endothelial cells. These dissolution products will also be used for animal toxicology testing. Scaffolds fabricated from this polymer will also be made radiopaque by iohexol coating in order to monitor the placement of the device in vivo.

For preclinical testing, a porcine model will be used. The 6 month implants will be evaluated for restenosis. Additionally, the results at 3 months will be combined with the histopathology for indications of efficacy. Earlier timepoints, e.g., 1 and 3 months, will demonstrate the extent to which the device enhances endothelial regeneration. Explant studies will use the methodology described by Pektok for a biodegradable small diameter vascular graft. Pektok et al., Circulation 2008, 118:2563-2570.

For clinical testing these devices will meet the safety and efficacy requirements of the FDA guidance for stents. Since this device releases a bioactive statin as part of the biodegradation process, it will meets the requirements of the Drug Eluting Stent (DES) guidance. (Prabhu et al, 2006; Guidance for Industry: Coronary Drug-Eluting Stents—Nonclinical and Clinical Studies, Draft Guidance, March 2008 DRAFT GUIDANCE Feb. 12, 2008; Guidance for Industry Coronary Drug-Eluting Stents—Nonclinical and Clinical Studies Companion Document DRAFT GUIDANCE March 2008; Farb 2008). The device will be compatible with stent delivery systems, processes and sterilization and meet the FDA Guidance for Industry: Coronary Drug-Eluting Stents—Nonclinical and Clinical Studies, Draft Guidance, March 2008, its companion document, and Non-Clinical Tests and Recommended Labeling for Intravascular Stents and Associated Delivery Systems, Jan. 13, 2005.

Human clinical studies will test vessel lumen patency and bioresorption characteristics of the scaffold over a one year period. In additional tests, devices will be used which elute a second generation mTOR agent such as everolimus or zotarolimus (or combination thereof) which help prevent restenosis. In some studies, the one stent will deliver two drugs, one targeting the endothelium and the other neointimal hyperplasia, both to the site of procedure related vessel lumen injury. Finally, studies looking at mortality from bleeding as result of reducing the duration of DAPT without a risk of stent thrombosis will be conducted. Currently, the DAPT study (NCT00977938) is looking at 12 vs. 30 months of DAPT.

The embodiments set forth herein were presented to best explain the present invention and its practical application and to thereby enable those of ordinary skill in the art to make and use the invention. However, those of ordinary skill in the art will recognize that the foregoing description and examples have been presented for the purposes of illustration. The description as set forth is not intended to be exhaustive or to limit the invention to the precise form disclosed. All references are incorporated by reference in their entirety. Many modifications and variations are possible in light of the teachings above without departing from the spirit and scope of the forthcoming claims.

Although the present invention has been disclosed above, the disclosure does not limit the present invention. Persons having ordinary skill in the art can make any changes or modifications without departing from the spirit and scope of the present invention. Consequently, the scope of protection of the present invention is based on the claims.

1. A polymer composition comprising:
a statin; and
one or more of lactide, glycolide, and ε-caprolactone,
wherein the statin is covalently attached to the one or more of lactide, glycolide and ε-caprolactone.

2. The composition of claim 1, wherein the polymer comprises a lactone containing statin and at least one lactide, glycolide and ε-caprolactone.

3. The composition of claim 2, wherein the lactide comprises about 45 wt. % to about 85 wt. % of the total polymer.

4. The composition of claim 2, wherein the glycolide comprises about 5 wt. % to about 50 wt. % of the total polymer.

5. The composition of claim 2, wherein the lactone containing statin comprises about 15 wt. % to about 25 wt. % of the total polymer.

6. The composition of claim 1, wherein the statin is one or more of simvastatin, atorvastatin, cerivastatin, fluvastatin, lovastatin, mevastatin, pitavastatin, pravastatin, rosuvastatin, simvastatin, velostatin, dalvastatin, carvastatin, and cefvastatin.

7. The composition of claim 1, further comprising one or more additional pharmaceutical agents selected from the group consisting of paclitaxel, mTOR, rapamicin, sirolimus, everolimus, and zotarolimus.

8. The composition of claim 1, further comprising one or more of poly(L-lactic acid) (PLA), poly(lactide-co-glycolide) (PLGA), poly(L-lactide) (PLLA), poly(ethylene glycol) (PEG), montmorillonite (MMT), poly(L-lactide-co-e-caprolactone) (P(LLA-CL)), poly(ε-caprolactone-co-ethyl ethylene phosphate) (P(CL-EEP)), poly[bis(p-methylphenoxy)phosphazene] (PNmPh), poly(3-hydroxybutyrate-co-3-hydroxyvalerate) (PHBV), poly(ester urethane) urea (PEUU), poly(p-dioxanone) (PPDO), polyurethane (PU), polyethylene terephthalate (PET), poly(ethylene-co-vinylacetate) (PEVA), poly(ethylene oxide) (PEO), poly(phosphazene), and poly(ethylene-co-vinyl alcohol).

9. A device comprising fibers, yarns, filaments, membranes, fabrics, meshes, or mesh-like structures prepared from a statin containing biocompatible polymer wherein at least one statin is covalently attached to the statin containing biocompatible polymer.

10. The device of claim 9, wherein the at least one statin is incorporated into a main chain of the statin containing biocompatible polymer.

11. The device of claim 9, wherein the at least one statin comprises an endgroup of the statin containing biocompatible polymer.

12. The device of claim 9, wherein the device is a stent.

13. The device of claim 9, wherein the polymer containing statin comprises a lactone containing statin and at least one lactide, glycolide, ε-caprolactone, or a combination thereof.

14. The device of claim 12, wherein the lactone containing statin comprises simvastatin or lovastatin.

15. A method of treating coronary artery disease comprising implanting a device comprising fibers, yarns, filaments, membranes, fabrics, meshes, or mesh-like structures prepared from a statin containing biocompatible polymer wherein at least one statin covalently is covalently attached to the statin containing biocompatible polymer to the tissue.

16. The method of claim 15, wherein scarring of said tissue is decreased.

17. The method of claim 15, wherein tissue adhesion is decreased.

18. The method of claim 15, wherein healing of said tissue is promoted.

19. The method of claim 15, wherein the statin is one or more of simvastatin, atorvastatin, cerivastatin, fluvastatin, lovastatin, mevastatin, pitavastatin, pravastatin, rosuvastatin, simvastatin, velostatin, dalvastatin, carvastatin, and cefvastatin.

20. The method of claim 15, further comprising one or more additional pharmaceutical agents selected from the group consisting of paclitaxel, mTOR, rapamicin, sirolimus, everolimus, and zotarolimus.
